(19) United States
(12) Patent Application Publication
(10) Pub. No.: US 2020/0315569 A1
(43) Pub. Date: Oct. 8, 2020

(54) SYSTEM AND METHOD FOR DETERMINING CONDITION OF FETAL NERVOUS SYSTEM

(71) Applicant: GENERAL ELECTRIC COMPANY, Schenectady, NY (US)

(72) Inventors: Suvadip Mukherjee, Bangalore (IN); Rahul Venkataramani, Bangalore (IN); Anuprriya Gogna, Bangalore (IN); Stephan Anzengruber, Zipf (AT)

(21) Appl. No.: 16/372,446

(22) Filed: Apr. 2, 2019

(51) Int. Cl. A61B 8/08; A61B 5/00

(52) U.S. Cl. CPC A61B 8/0866; A61B 5/0444; A61B 5/4362; A61B 8/523

(57) ABSTRACT

A method for determining a nervous system condition includes obtaining an estimate of a first scan plane among a plurality of planes of a maternal subject using a first deep learning network during a guided scanning procedure. The method further includes receiving a three-dimensional (3D) ultrasound volume corresponding to the initial estimate and determining an optimal first scan plane from the first deep learning network. The method further includes determining at least one of a second scan plane, a third scan plane and a fourth scan plane among the plurality of planes, based on the optimal first scan plane and at least one of a clinical constraint corresponding to the plurality of planes using a second deep learning network. The method includes determining a biometric parameter corresponding to nervous system based on at least one of the plurality of planes using a third deep learning network.

### BACKGROUND

Embodiments of the present specification relate generally to ultrasound imaging, and more particularly to systems and methods of acquiring scanning data and processing the acquired scanning data for diagnostic purposes in an efficient manner. Embodiments of the present specification are explained in the context of assessing condition of fetal nervous system.

Typically, an ultrasound imaging technique includes emission of an ultrasound beam towards a determined portion in a human body, for example, a fetus, a kidney, and the like, and the reflected beam is processed to obtain an image associated with a section of soft tissue or the bloodstream. An ultrasound system has advantages of being small, inexpensive, displayable in real time, and safe as the subject is not exposed to an X-ray and other harmful radiations.

Ultrasound image technique is commonly used to determine health of a fetus during pregnancy. Specifically, a chromosomal abnormality in a fetus is generally identified by measuring a geometrical parameter such as a thickness of a nuchal translucency (NT) of the fetus. Presence of a thick NT determines a Down's syndrome, or other chromosomal abnormalities such as a deformity of the heart or a Turners syndrome. The same chromosomal abnormality may also be determined by measuring a variety of geometric parameters using ultrasound imaging. In the case of identifying Down's syndrome in a fetus, an angle between the palate and the dorsum nasi, namely, the front maxillary facial (FMF) angle, may be measured. Alternatively, Down's syndrome may also be determined based on measuring the biparietal diameter (BPD), head circumference (HC), abdominal circumference (AC), femur length (FL), and the like. A gestational age and a weight of the fetus may be estimated based on the measured geometrical parameters.

Measurement of geometrical parameters of the fetus requires obtaining an accurate sagittal, transversal and other standard scan plane views from ultrasound data. Conventionally, the scan plane views are determined based on experience of the physician. Consequently, it is plausible that the measured thickness of the NT of the fetus or the FMF angle between the palate and the dorsum nasi may have some errors and may be different from actual values. Thereby, resulting in difficulties in making an accurate diagnosis. Occasionally, semiautomated techniques are employed in determining the scan planes and corresponding geometric parameters, in which human intervention is required to complete the assessment of the fetal health. However, such techniques are affected by variability of anatomy of patients as well as variations introduced by an operator.

### BRIEF DESCRIPTION

In accordance with one aspect of the present specification, a method is disclosed. The method includes obtaining an initial estimate of a first scan plane corresponding to a fetus of a maternal subject using a first deep learning network during a guided scanning procedure. The scan plane includes one of a trans thalamic plane (TTP), a trans-ventricular plane (TVP), a mid-sagittal plane (MSP) and/or a trans-cerebellar plane (TCP). The method further includes receiving a three-dimensional (3D) ultrasound volume of the fetus corresponding to the initial estimate of the first scan plane. The method also includes determining an optimal first scan plane from the first deep learning network based on the 3D ultrasound volume and the initial estimate of the first scan plane. The method further includes determining at least one of a second scan plane, a third scan plane and/or a fourth scan plane based on the 3D ultrasound volume, the optimal first scan plane and at least one of a clinical constraint corresponding to the TTP, the TVP, the MSP and/or the TCP using a corresponding second deep learning network. Each of the second scan plane, the third scan plane and the fourth scan plane include one of the TTP, the TVP, the MSP and/or the TCP and distinctly different from the first scan plane. The method includes determining a biometric parameter corresponding to nervous system of the fetus based on at least one of the first scan plane, the second scan plane, the third scan plane, and/or the fourth scan plane and the clinical constraint using a third deep learning network. The method also includes determining a nervous system condition of the fetus based on the biometric parameter.

In accordance with another aspect of the present specification, a system is disclosed. The system includes an ultrasound scanning probe configured to obtain an initial estimate of a first scan plane corresponding to a fetus of a maternal subject using a first deep learning network during a guided scanning procedure. The scan plane comprises one of a trans thalamic plane (TTP), a trans-ventricular plane (TVP), a mid-sagittal plane (MSP) and/or a trans-cerebellar plane (TCP). The system further includes a data acquisition unit communicatively coupled to the ultrasound probe and configured to receive scan data obtained by the ultrasound scanning probe. The system also includes a learning unit communicatively coupled to the data acquisition unit and configured to receive, from the data acquisition unit, a three-dimensional (3D) ultrasound volume of the fetus corresponding to the initial estimate of the first scan plane. The learning unit is further configured to determine an optimal scan plane from the first deep learning network based on the 3D ultrasound volume and the initial estimate of the first scan plane. The learning unit is also configured to determine at least one of a second scan plane, a third scan plane and/or a fourth scan plane based on the 3D ultrasound volume, the optimal first scan plane and at least one of a clinical constraint corresponding to the TTP, the TVP, the MSP and/or the TCP using a corresponding second deep learning network. Each of the second scan plane, the third scan plane and/or the fourth scan plane include one of the TTP, the TVP, the MSP and/or the TCP and distinctly different from the first scan plane. The learning unit is also configured to determine a biometric parameter corresponding to nervous system of the fetus based on at least one of the first scan plane, the second scan plane, the third scan plane, and/or the fourth scan plane and the clinical constraint using a third deep learning network. The system also includes a diagnosis unit communicatively coupled to the learning unit and configured to determine a nervous system condition of the fetus based on the biometric parameter.

A non-transitory computer readable medium having instructions to enable at least one processor unit to obtain an initial estimate of a first scan plane corresponding to a fetus of a maternal subject using a first deep learning network during a guided scanning procedure. The scan plane comprises one of a trans thalamic plane (TTP), a trans-ventricular plane (TVP), a mid-sagittal plane (MSP) and/or a trans-cerebellar plane (TCP). The instructions also enable the at least one processor to receive a three-dimensional (3D) ultrasound volume of the fetus corresponding to the initial estimate of the first scan plane. Further, the instructions also enable the at least one processor to determine an optimal scan plane from the first deep learning network based on the 3D ultrasound volume and the initial estimate of the first scan plane and determine at least one of a second scan plane, a third scan plane and/or a fourth scan plane based on the 3D ultrasound volume, the optimal first scan plane and at least one of a clinical constraint corresponding to the TTP, the TVP, the MSP and/or the TCP using a corresponding second deep learning network. Each of the second scan plane, the third scan plane and/or the fourth scan plane include one of the TTP, the TVP, the MSP and/or the TCP and distinctly different from the first scan plane. The instructions further enable the at least one processor to determine a biometric parameter corresponding to nervous system of the fetus based on at least one of the first scan plane, the second scan plane, the third scan plane, and/or the fourth scan plane and the clinical constraint using a third deep learning network and determine a nervous system condition of the fetus based on the biometric parameter.

### DRAWINGS

These and other features and aspects of embodiments of the present invention will become better understood when the following detailed description is read with reference to the accompanying drawings in which like characters represent like parts throughout the drawings, wherein;

FIG. 1 is a diagrammatic illustration of a system for determining a nervous system condition in a fetus in accordance with an exemplary embodiment;

FIG. 2 is an image illustrating selection of a trans-thalamic plane (TTP) in accordance with an exemplary embodiment;

FIGS. 3A-3C are images illustrating selection of scan planes of the fetal brain in accordance with an exemplary embodiment;

FIG. 4 is an image illustrating selection of a trans-ventricular plane (TVP) in accordance with an exemplary embodiment;

FIG. 5 is an image illustrating selection of a trans-cerebellar plane (TCP) in accordance with an exemplary embodiment;

FIG. 6 is a schematic of a workflow for determining a condition of a nervous system of a fetus in accordance with an exemplary embodiment; and

FIG. 7 is a flow chart of a method for determining a condition of a nervous system of a fetus in accordance with an exemplary embodiment.

### DETAILED DESCRIPTION

As will be described in detail hereinafter, systems and methods for ultrasound imaging are presented. More particularly, the systems and methods are configured to enable an operator to acquire scanning data for diagnostic purposes in an efficient manner. Embodiments of the present specification are explained in the context of assessment of a fetal nervous system using three-dimensional (3D) ultrasound image dataset.

The terms “sagittal plane,” “coronal plane” and “transverse plane” refer respectively to lateral, frontal and axial planes in a three-dimensional anatomy of a subject. The sagittal plane divides the body into a left portion and a right portion, the coronal plane divides the body into a front portion and a back portion and the transverse plane divides the body into an upper portion and a bottom portion. The back portion is also referred to as a “dorsal portion” or “posterior portion” and the front portion is also referred to as a “ventral portion” or an “anterior portion”. A sagittal plane that divides the body into equal left and right portions is termed as ‘mid sagittal plane’ and is abbreviated as MSP. The top portion is referred to as a “superior portion” or “cranial portion” and the bottom portion is also referred to as an “inferior portion” or a “caudal portion.” The prefix “trans” is generally used with an anatomical structure in a 3D volume of an organ of interest to refer to a plane associated with the anatomical structure. For example, the term “trans-ventricular plane” abbreviated herein as TVP includes the anterior and posterior portions of the lateral ventricles. The anterior portion of the lateral ventricles (frontal or anterior horns) appear as two comma-shaped fluid filled structures and have a well-defined lateral wall and are medially separated by a cavum septi pellucidi (CSP). The term “trans-thalamic plane” abbreviated as TTP includes thalami and hyppocampal gyrus. The term “trans-cerebellar plane” abbreviated as TCP is related to cerebellum portion and cistena magna. The term “mid sagittal plane” refers to a plane along the sagittal suture. The term “para sagittal plane” refers to a plane that divides the body into left and right portions, parallel to the sagittal plane. Sometimes, the term para sagittal plane also refers to a plane angularly separated from the sagittal plane. The term “fetal sonography” is used to refer to evaluation of conditions of central nervous system (CNS) of a fetus using ultrasound images acquired along axial and sagittal planes.

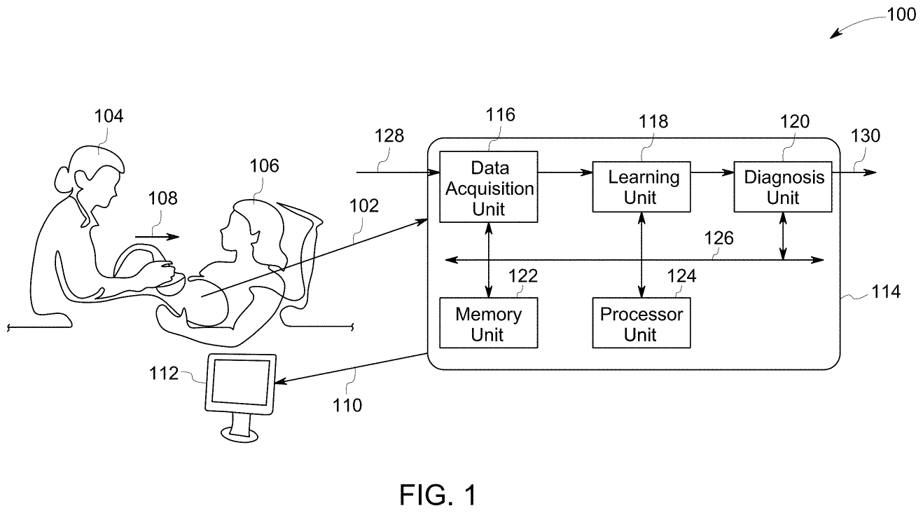

FIG. 1 is a diagrammatic illustration of an ultrasound scanner 100 for determining a medical condition related to a subject 106. In a specific example, the subject 106 may be an expecting woman evaluated during a trimester pregnancy to assess fetal health, to monitor the development of the fetal brain, or both, for example, through fetal sonography. The ultrasound scanning is used for evaluation of conditions of central nervous system (CNS) of the fetus in accordance with exemplary embodiments of the present specification. The ultrasound scanner 100 includes an ultrasound scanning probe 108 used by an operator 104 to examine the subject 106 and generate ultrasound scanning data, generally represented by reference numeral 102. The ultrasound scanner 100 further includes a data processing system 114 communicatively coupled to the ultrasound scanning probe 108 and configured to receive the ultrasound scanning data 102. The data processing system 114 is further configured to generate an output data, generally represented by reference numeral 110, based on the ultrasound scanning data 102. In one embodiment, the output data 110 may be in the form of a feedback to the operator 104 to make modifications or adjustments to the scanning operation to enable the scanning operation to be performed more accurately. In another embodiment, the output data 110 may be an image data presentable to the operator 104. In yet another embodiment, the output data 110 may be a diagnostic information corresponding to a diagnostic condition of an organ of interest of the subject 106. In one non-limiting example, the diagnostic condition may be a hypoplasia condition representative of underdevelopment or incomplete development of fetal central nervous system. In another non-limiting example, the diagnostic condition may be a dysplasia condition representative of an anomalistic development of fetal central nervous system. The ultrasound scanner 100 also includes an output device 112 for presenting the output data 110 to the operator 104. The output device 112 may include a monitor, a speaker, a tactile device, or other devices.

In the illustrated embodiment, the data processing system 114 includes a data acquisition unit 116, a learning unit 118, a diagnostic unit 120, a memory unit 122 and a processor unit 124 coupled to each other via a communications bus 126. In one embodiment, each of the units 116, 118, 120, 122, 124 may include at least one processing element such as a processor or a controller, one or more memory chips, at least one input lead for receiving input data required by the respective unit and at least one output lead for providing output data from the respective unit to one or more other units or devices. Further, each of the units 116, 118, 120, 122, 124 may further include circuitry to interface with one or more of the other units, the ultrasound scanning probe 108, the output device 110 and a user input, generally represented by reference numeral 128.

In an exemplary embodiment, the ultrasound scanning probe 108 is configured to obtain an initial estimate of a first scan plane corresponding to a fetus of a maternal subject in a guided scanning procedure. The learning unit 118 is configured to provide guidance while an operator is acquiring the initial estimate. The first scan plane comprises one of a MSP, TTP, TVP and TCP. The learning unit 118 is configured to receive a plane type as an input and estimate quality of the specified scan plane as the ultrasound scanning probe is moved while examining the maternal subject. The operator is enabled to identify a good estimate of the specified scan plane as the first scan plane either based on the estimated quality or based on his/her experience or both. Further, the ultrasound scanning probe 108 is configured to acquire a three-dimensional (3D) ultrasound volume corresponding to the initial estimate of the first scan plane either in a semi-automatic manner or completely in an automatic fashion. The acquired 3D ultrasound volume is referred herein as the ‘ultrasound scanning data’.

The data acquisition unit 116 is communicatively coupled to the ultrasound scanning probe 108 and configured to receive the ultrasound scanning data 102. The ultrasound scanning data 102 includes the 3D ultrasound volume corresponding to a fetus of a maternal subject. The data acquisition unit 116 may include necessary circuitry to interface with the ultrasound scanning probe 108 and interpret the ultrasound scanning data 102 as image frames. The data acquisition unit 116 is also configured to receive user input 128 from an operator console such as, but not limited to key board, or a touch display. The data acquisition unit 116 is also configured to transfer the ultrasound scanning data 102 to and retrieve historical data from the memory unit 122. The data acquisition unit 116 is also configured to receive the initial estimate of the first scan plane from the ultrasound scanning probe 108.

The learning unit 118 is communicatively coupled to the data acquisition unit 116 and configured to receive an initial estimate of the first scan plane. The learning unit 118 includes one or more learning networks, machine learning modules configured to learn and estimate scan planes, biometric parameters associated with the scanning planes and nervous system conditions. In one embodiment, the learning unit 118 is configured to assist the operator to select a good initial estimate of the first scan plane. The learning unit 118 employs a first deep learning network for providing a quality indicator of the scan plane acquired by the ultrasound probe 108. Further, the learning unit 118 is also configured to generate a plurality of estimates of the first scan plane as first scan plane candidates based on the initial estimate. The plane parameters of the initial estimate of the first scan plane is varied within a pre-determined range of values to generate plane parameters of the plurality of first scan plane candidates. The learning unit 118 is further configured to determine an optimal first scan plane candidate from the plurality of first scan plan candidates using the first deep learning network. Specifically, the first deep learning network is configured to determine a quality score corresponding to each of the plurality of first scan plane candidates and generate a plurality of quality scores. Further, a minimum score among the plurality of quality scores may be determined. In one embodiment, the learning unit 118 is configured to select a first scan plane candidate among the plurality of first scan plane candidates corresponding to the minimum score. The learning unit 118 is also configured to determine at least one of a second scan plane, a third scan plane and a fourth scan plane based on the 3D ultrasound volume, the optimal first scan plane using a corresponding second deep learning network. Each of the second scan plane, the third scan plane and the fourth scan plane is one of the MSP, the TTP, the TVP and/or the TCP and distinctly different from the first scan plane. It may be noted herein that each of the first scan plane, the second scan plane, the third scan plane and/or the fourth scan plane are uniquely mapped to the MSP, the TTP, the TVP and/or the TCP. Determination of planes by the second learning network is based on specified clinical guidelines used in practice. As an example of the clinical guidelines, the MSP is determined based on anatomy or geometrical properties related to the anatomy within the maternal subject. As another example of the clinical guidelines, the MSP is constrained to be orthogonal to the TTP and parallel to TVP. As yet another example of the clinical guidelines, the TCP is constrained to be orthogonal to MSP and parallel to TTP. Similarly, it may be noted that the clinical guideline also constrains the TVP to be parallel to the TCP.

In first embodiment, the first scan plane corresponds to the TTP, the second plane corresponds to the mid-sagittal plane (MSP), the third scan plane corresponds to the trans-cerebellar plane (TCP) and the fourth scan plane corresponds to the trans-ventricular plane (TVP). In a second embodiment, the first scan plane corresponds to the TVP, the second scan plane corresponds to the TTP, the third scan plane corresponds to the MSP and the fourth scan plane corresponds to the TCP. In a third embodiment, the first scan plane corresponds to the TCP, the second scan plane corresponds to the MSP, the third scan plane corresponds to the TTP and the fourth scan plane corresponds to the TVP. In a fourth embodiment, the first scan plane corresponds to the MSP, the second scan plane corresponds to the TCP, the third scan plane corresponds to the TTP and the fourth scan plane corresponds to the TVP.

Specifically, in the first embodiment, the learning unit 118 is configured to segment the optimal TTP to detect a midline of the cranium and a midpoint of TTP based on the segmented optimal TTP. The learning unit 118 is also configured to determine a plane parameter vector corresponding to the MSP based on the midline of the cranium. The learning unit 118 is further configured to generate the MSP from the determined plane parameter. In a further embodiment, the learning unit 118 is configured to generate a plurality of estimates of the TVP as TVP candidates. In a specific embodiment, the plurality of TVP candidates are generated by varying the plane parameters of the optimal TTP and the plane parameters of the MSP such that each of the plurality of TVP candidates is parallel to the optimal TTP and orthogonal to the MSP. In such an embodiment, the learning unit 118 is further configured to receive the second deep learning network configured to determine an optimal TVP. Also, the learning unit 118 is configured to estimate an optimal TVP by processing the plurality of TVP candidates by the second deep learning network. The second deep learning network is configured to generate a plurality of quality scores corresponding to the plurality of TVP candidates. Each of the plurality of quality scores is representative of proximity of corresponding TVP candidate with the desired TVP in the 3D volume. A minimum score among the plurality of quality scores is selected by the learning unit 118 and a corresponding TVP candidate is identified as the optimum TVP.

Further, in the first embodiment, the learning unit 118 is configured to generate a plurality of estimates of the TCP as TCP candidates. The plurality of TCP candidates may be generated by varying the plane parameters of the optimal MSP and the optimal TTP parameters within a predetermined range of values. The plurality of TCP candidates is generated such that each of the plurality of TCP candidates is either orthogonal to the optimal MSP or oriented to a perpendicular to the optimal TTP by angle within a prespecified angular span. Further, the learning unit 118 is configured to estimate an optimal TCP by processing the plurality of TCP candidates using the second deep learning network. In such a case, the second learning network is further configured to determine an optimal TCP. The learning unit 118 is also configured to determine a biometric parameter corresponding to nervous system of the fetus based on at least one of the MSP, TCP and TVP and a geometric constraint using a third deep learning network. In one embodiment, the learning unit 118 is configured to determine at least one of a head circumference (HC), a biparietal diameter (BPD), an occipito-frontal diameter (OFD), a trans-cerebellar diameter (TCD), a cisterna magna (CM), and a posterior ventricle (Vp) based on one or more of the optimal TTP, the MSP, the TCP and the TVP.

The diagnosis unit 120 is communicatively coupled to the learning unit 118 and configured to determine a nervous system condition of the fetus based on the biometric parameter. In the FIG. 1, the nervous system condition is generally represented by reference numeral 130. Further, the diagnosis unit 120 is configured to determine an object segmentation using a fourth deep learning network. The fourth deep learning network is trained to do perform image segmentation using a plurality of annotated images. Alternatively, the fourth deep learning network may be trained to determine the location (or presence) of an anatomical structure without segmentation. Specifically, the fourth deep learning network may be trained to use a landmark detection network or a classification network that classifies healthy images from one or more pathological images. In a further embodiment, the diagnosis unit 120 is configured to identify a location on the segmented image and performing automated measurements using a caliper placement algorithm. In one embodiment, the diagnosis unit 120 is configured to compare the biometric parameter with a pre-determined threshold and choose a diagnostic option based on the comparison. In one embodiment, the option refers to a nervous system condition and a category associated with the nervous system condition. Further, the option may include an action such as displaying the nervous system condition and printing the category on a display device.

In the second embodiment, the learning unit 118 is configured to determine an optimal TVP as the first scan plane. Further, the learning unit 118 is configured to generate, a plurality of TTP candidates parallel to the optimal TVP. The learning unit 118 is also configured to estimate midline from falx in TTP and place MSP orthogonally through midline. The learning unit 118 is configured to determine a plurality of TCP candidates in a space orthogonal to MSP. The learning unit 118 is configured to determine a plane which is rotated about thirty five degrees from the TTP about midpoint of falx with a parallel shift to determine the TCP.

In the third embodiment, the learning unit 118 is configured to determine an optimal TCP as the first scan plane. Further, the learning unit 118 is configured to generate, a plurality of MSP candidates parallel to the optimal TCP. The learning unit 118 is also configured to estimate midline from falx in TCP and place MSP orthogonally through midline. The learning unit 118 is configured to determine a plurality of TTP candidates in a space orthogonal to MSP. The learning unit 118 is configured to determine a plane which is rotated about thirty five degrees from the TCP about midpoint of falx with a parallel shift to determine the TTP. The learning unit 118 is also configured to determine the TVP so as to be parallel to the TTP.

In the fourth embodiment, the learning unit 118 is configured to determine an optimal MSP as the first scan plane. Further, the learning unit 118 is configured to determine the TCP using anatomy based techniques and geometric constraints among the plurality of planes. Specifically, in one embodiment, the learning unit 118 is configured to determine locations of at least one of a cerebellum and cavum septi pellucidi based on the optimal MSP. Further, a plurality of TCP candidates is determined by the learning unit 118. A plane among the plurality of TCP candidates that is orthogonal to at least one of the cerebellum and cavum septi pellucidi is considered as the required TCP. The learning unit 118 is also configured to determine the TTP that is parallel to TCP and rotated about a midpoint of falx of the TCP. Finally, the learning unit 118 is also configured to determine a TVP that is parallel to TCP.

The processor unit 124 is communicatively coupled to the memory unit 122 and configured to perform control operations for the data acquisition unit 116, the learning unit 118 and the diagnosis unit 120. The processor unit is also configured to control the storage and retrieval of data in/out of the memory unit 120. In some embodiments, the processor unit 124 may also assist in performing or may perform functionality of the data acquisition unit 116, the learning unit 118 and the diagnosis unit 120. The processor unit 124 includes a graphical processing unit (GPU), one or more microprocessors, and a microcontroller. The processor unit 124 further includes specialized circuitry or hardware such as, but not limited to, a field programmable gate array (FPGA), application specific integrated circuit (ASIC). Although the processor unit 124 is illustrated as a single processor, a plurality of computing elements co-located or distributed in multiple locations and configured to co-operatively operate may be used. In an alternative embodiment, the processor unit 124 may be a cloud service or any other computation as a service mechanism.

The memory unit 122 is communicatively coupled to the data acquisition unit 116 and configured to store the ultrasound scanning data 102. Further, the memory unit 122 is also configured to receive user input 128 provided by an operator during the scanning, or ultrasound scanning parameters that are set at the beginning of the scanning procedure. The memory unit 120 may be further configured to provide inputs to the learning unit 118 and store the outputs in the diagnosis unit 120. The memory unit 120 may be a single memory storage unit or a plurality of smaller memory storage units coupled together to work in a coordinated manner. In one embodiment, the memory unit 120 may be a random-access memory (RAM), read only memory (ROM), or a flash memory. The memory unit 122 may also include, but not limited to, discs, tapes, or hardware drive based memory units. It may be noted that a part of the memory unit 122 may also be disposed at a remote location as a hardware unit or as a cloud service providing computational and storage services. In one embodiment, the memory unit 122 may be pre-loaded with deep learning models, training data in the form of labelled anatomical information, and historical image data. In some embodiments, the training data may be labelled with a plurality of attributes such as, but not limited to, age, region, gender and medical conditions of subjects.

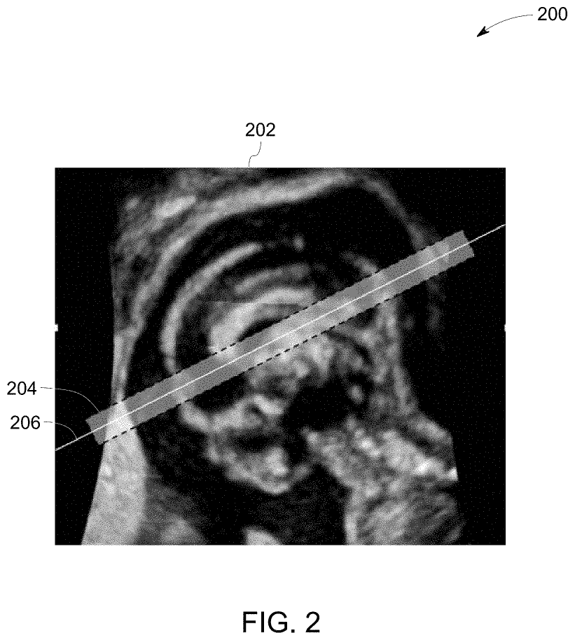

FIG. 2 is an image 200 illustrating selection of a trans-thalamic plane (TTP) in accordance with the first embodiment. The image 200 includes a plurality of TTP candidates 204 generated by a learning unit, such as the learning unit 118 of FIG. 1, using an initial TTP estimate. The image 200 also includes the TTP candidate 206 selected from the plurality of TTP candidates 204. The TTP candidate 206 is selected such that the TTP candidate 206 has a minimum score among the plurality of quality scores that are generated corresponding to the plurality of TTP candidates. The plurality of quality scores may be generated by processing each of the plurality of candidates 204 using the first deep learning network. The first deep learning network may be retrieved from the memory unit 122 for generating the plurality of quality scores. In one embodiment, the first deep learning network is generated by training a neural network using labelled ultrasound images stored in the memory unit 122. The labelling information in this embodiment includes a numerical value representative of a separation of a TTP candidate from the desired TTP plane. Lower numerical values indicate proximity to the desired TTP candidate and the larger numerical values represent increased distance of the TTP candidate from the desired TTP plane. It may also be noted that, in an alternative embodiment, a maximum score among the plurality of quality scores may be chosen when the maximum numerical scores indicate proximity of TTP candidate with the desired TTP candidate. The training is performed in an off-line mode of the ultrasound scanner and the trained deep learning network is stored in the memory unit 122.

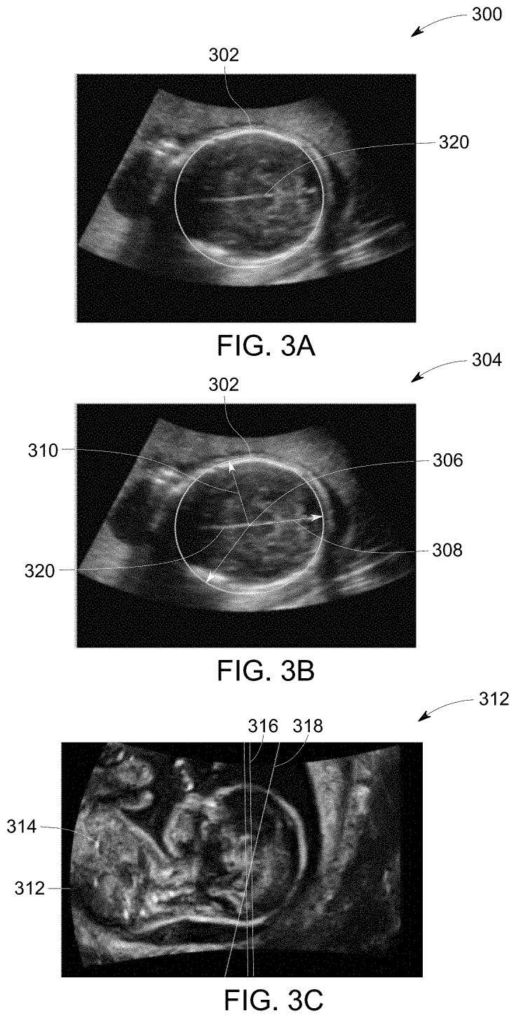

FIG. 3A-3C are images 300, 304, 312 illustrating selection of scan planes of the fetal brain in accordance with the first embodiment. The image 300 and 304 corresponds to TTP candidates such as the one referred by numeral 206 in FIG. 2 and the image 312 corresponds to a mid-sagittal plane (MSP). The image 300 illustrates a cranium 302, a large sickle-like crescent-shaped fold 320 of meningeal layer of dura mater that descends vertically in the longitudinal fissure between the cerebral hemispheres of the human brain. The cranium 302 is determined for the selected TTP candidate 206 using a segmentation technique applied on the image 300. The image 304 is a replica of the TTP candidate image 300 illustrating cranium midline. In the image 304, the midline falx 308 and a midpoint 306 of the selected TTP candidate 206 are also illustrated in the image 304. The midpoint 306 is also determined using the segmented cranium image 302. A normal 310 to the MSP is illustrated in the image 304. Further, plane parameters for the MSP are determined based on the detected midline falx 308 and the normal line 310 using analytical equations. Finally, the MSP is generated from the computed parameters. The image 312 illustrating texture map 314 corresponds to the generated MSP. A perpendicular line 316 in the image MSP illustrates the TTP plane corresponding to the image 300.

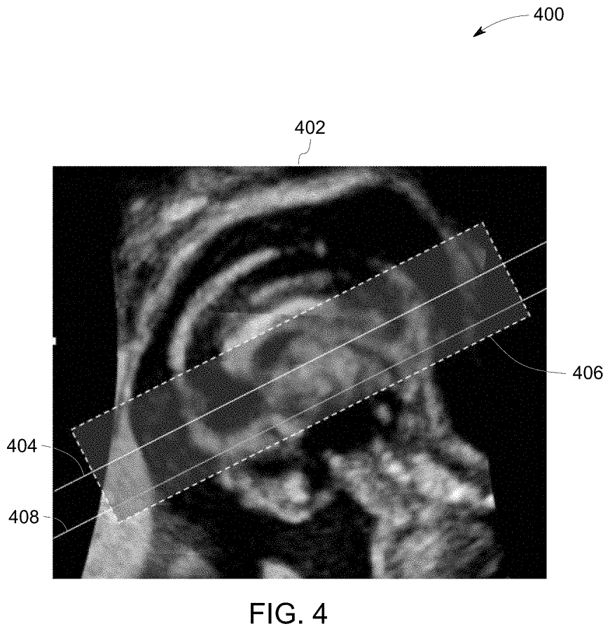

FIG. 4 is a FIG. 400 illustrating selection of a trans-ventricular plane (TVP) in accordance with the first embodiment. The FIG. 400 includes an image 402 representative of a fetal brain obtained during the ultrasound scanning of an expectant mother. The image 402 includes the TTP candidate 404 selected using the first deep learning network as explained with reference to FIG. 2. The image 402 also includes a plurality of TVP candidates 406 generated by the learning unit 118 of FIG. 1. The plurality of TVP candidates 406 are selected such that the selected TVP candidates are parallel to the selected TTP candidate 304 and orthogonal to the MSP. The image 402 also includes an optimal TVP candidate 408 selected by evaluating the plurality of TVP candidates 406 using the second learning network. In one embodiment, the evaluating the plurality of TVP candidates 406 includes processing each of the plurality of TVP candidates 406 by the second deep learning network to generate a second plurality of quality scores. The quality scores generated by the second learning network are representative of proximity of the plurality of TVP candidates with the optimal TVP candidate. In one embodiment, smaller scores correspond to TVP candidates similar to the optimal TVP candidate. Further, a minimum value among the plurality of second quality scores is selected. A TVP candidate among the plurality of TVP candidates corresponding to the minimum value is selected as the optimal TVP candidate 408. It may be noted herein that in an alternate embodiment, a maximum value among the plurality of second quality scores may be selected to determine the optimal TVP candidate 408. In one embodiment, the second deep learning network is retrieved from the memory unit 122. In some embodiments, the second deep learning network is trained offline by the learning unit 118 using a training data set having labelled ultrasound images stored in the memory unit.

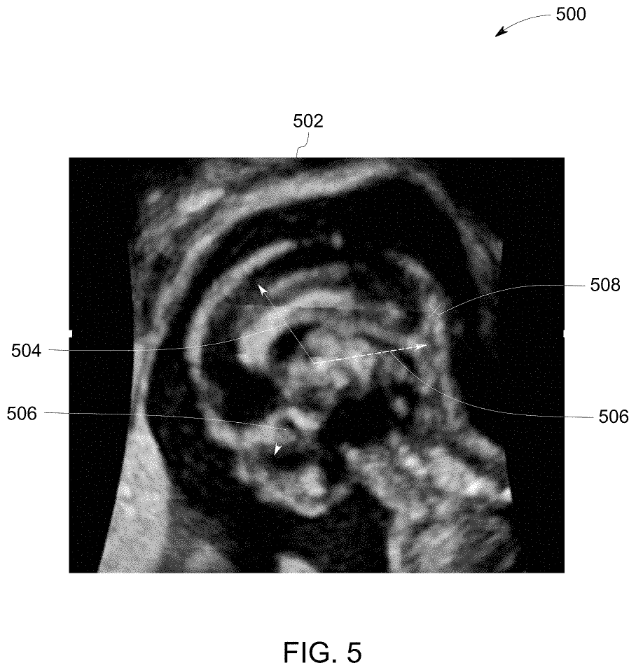

FIG. 5 is an image 500 illustrating selection of a TCP in accordance with first embodiment. The TCP plane is selected from a plurality of TCP candidate planes which are orthogonal to MSP plane. Additionally, the plurality of TCP candidates is also selected such that the TCP candidates are oriented with respect to a TTP normal 504 by an angle within a prespecified angular span. In the illustrated embodiment, the MSP normal 506 is illustrated. The image 500 includes a cross product (not shown in FIG. 5) of the TTP normal 504 and the MSP normal 506. A TCP candidate is selected from the plurality of TCP candidates using a third deep learning network. In one embodiment, each of the plurality of TCP candidates are processed using the third deep learning network to generate a plurality of third quality scores. A minimum value among the third quality scores is determined and a corresponding TCP candidate is selected as the optimal TCP. The third deep learning network may be retrieved from the memory unit 122. In one embodiment, the third deep learning network may be trained offline by the learning unit 118 using the training dataset stored in the memory unit 122. The training dataset includes a plurality of labelled ultrasound images annotated by an experienced medical professional and verified for clinical accuracy. It may be noted that in some embodiments, the second deep learning network may be further trained to select the optimal TCP candidate from the plurality of TCP candidates.

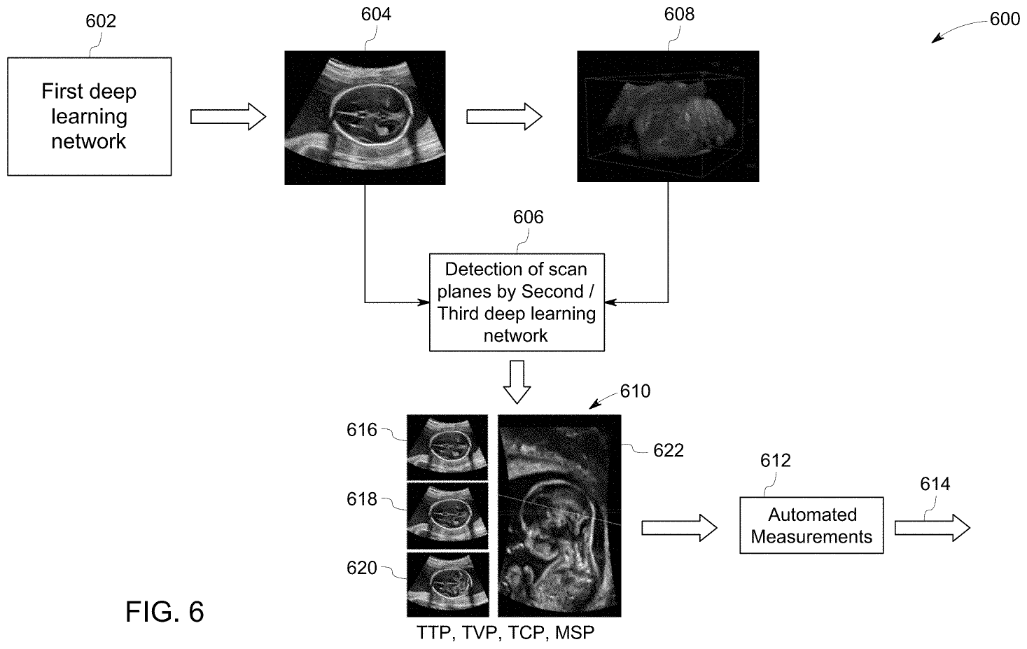

FIG. 6 is a schematic 600 illustrating a workflow for determining a condition of a nervous system of the fetus in accordance with the first embodiment. The schematic 600 illustrates providing artificial intelligence guidance to operator of an ultrasound scanner for initiating acquisition of 3D volume data 608 using a first deep learning network 602. Specifically, the first deep learning network 602 provides a confidence score for different plane locations acquired as the operator navigates freely over the organ of interest. The artificial intelligence guidance is based on a deep learning configured to generate the confidence score indicative of acceptability of present scan plane for initialization of acquisition of 3D volume data 608. In one embodiment, the confidence score is generated in real-time by the learning unit 118. The artificial intelligence guidance assists the operator to reach an initial TTP 604 in the neighborhood of the optimal TTP. The initial TTP 604 is determined by comparing the quality scores generated by the first deep learning network 602 with a prespecified range of quality scores. In an alternative embodiment, the artificial intelligence guidance is based on an image segmentation technique. In such an embodiment, the first deep learning network 602 is a segmentation network configured to assess presence of an anatomical structure of interest within the plurality of TTP candidates. The initial TTP 604 is the one image among the plurality of TTP candidates that includes maximum portion of the anatomical structure of interest. The initial TTP 604 in the neighborhood of the optimal TTP is used by the second (and third) deep learning network for identification of the scan planes, namely the TVP and the TCP.

The schematic 600 further illustrates generation of four scan planes using a second deep learning network at step 606. In one embodiment the second deep learning network is trained to generate all the four scan planes TTP, TVP, TCP and MSP. In another embodiment, a third deep learning network is also used at step 606. In such an embodiment, where the third deep learning network is also used, the second deep learning network may be trained to determine the TTP and the MSP. In an alternate embodiment, the MSP is determined based on segmented TTP using geometric computations. The third deep learning network may be trained to determine the TVP and TCP scan planes. The schematic 600 illustrates all the four scan planes TTP 616, MSP 618, TVP 620 and TCP 622 in image 610.

The schematic 600 also includes an automated measurement step 612 where image segmentation, measurement of parameters, and diagnostic decision making are performed using a fourth deep learning network. In one embodiment, the fourth deep learning network is trained to process one or more of the TTP, MSP, TVP, TCP for performing image segmentation. In another embodiment, a separate deep learning network is trained to process each of the TTP, MSP, TVP and TCP to generate respective segmented images. In one embodiment, the fourth deep learning network is trained using a batch of pixel-level annotated images. The segmentation output is refined using image analysis techniques such as, but not limited to, morphological filters and grayscale filters. The segmentation refinement may also be performed using classical unsupervised image processing techniques such as, but not limited to, vesselness filter and grayscale morphology. Further, one or more automated measurement using, but not limited to, caliper placement technique and co-ordinate based measurements may be used to determine one more diagnostic parameters. In one embodiment, the diagnostic parameters are compared with suitable threshold values to determine a fetal brain condition. In one embodiment, the caliper placement algorithm uses unsupervised approaches to identify the object orientation, and alignment, and can automatically predict the positions where clinical measurements are made.

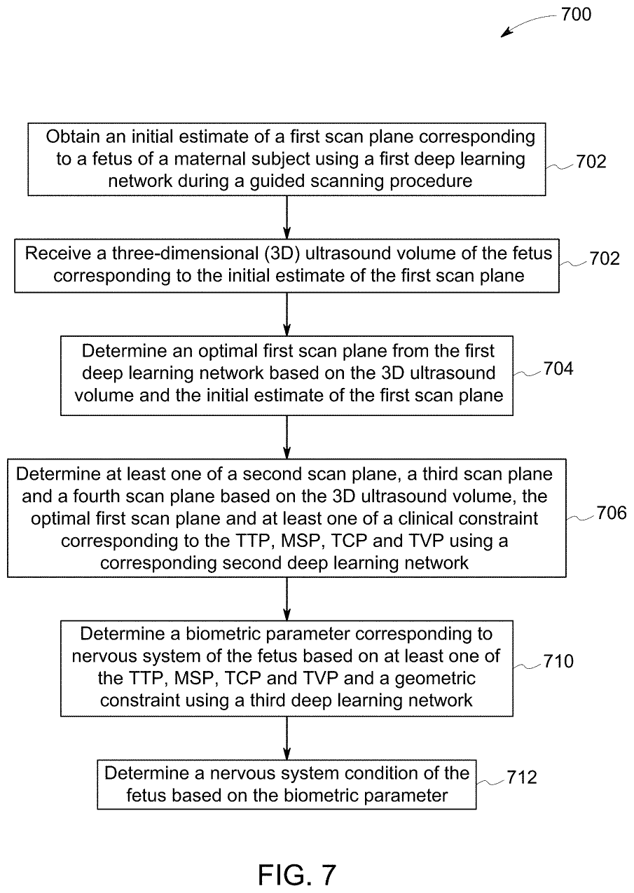

FIG. 7 is a flow chart 700 of a method for determining a condition of nervous system of the fetus in accordance with an exemplary embodiment. The method includes obtaining an initial estimate of a first scan plane corresponding to a fetus of a maternal subject using a first deep learning network during a guided scanning procedure as illustrated in step 702. It may be noted herein that an experienced operator may manually obtain the initial estimate of the first scan plane by moving the ultrasound probe over an anatomy of interest. Alternatively, an inexperienced operator may receive guidance from the first deep learning network to select the initial estimate while moving the ultrasound probe over the anatomy of interest. The method further includes receiving a 3D ultrasound volume of the fetus of the maternal subject corresponding to the initial estimate as illustrated in step 704. In step 706, the method also includes determining an optimal first scan plane from the first deep learning network based on the 3D ultrasound volume and the initial estimate of the first scan plane. In one embodiment, determining the optimal first scan plane includes generating a plurality of candidates for the first scan plane based on the initial estimate.

Further, the determining operation of step 706 also includes determining a quality score corresponding to each of the plurality of candidates for the first scan plane using the first deep learning network to generate a plurality of quality scores. Determining the optimal first scan plane further includes determining a minimum score among the plurality of quality scores. Determining the optimal first scan plane also includes selecting a first scan plane candidate among the plurality of candidates for the first scan plane corresponding to the minimum score. In some embodiments, a maximum score among the plurality of quality scores may be used to determine the optimal first scan plane. It may be noted herein that the first scan plane may include any one of the TTP, TCP, TVP and MSP.

The method further includes determining at least one of a second scan plane, a third scan plane and/or a fourth scan plane based on the 3D ultrasound volume, the optimal first scan plane and at least one of a clinical constraint corresponding to the second scan plane, the third scan plane and the fourth scan plane using a corresponding second deep learning network as illustrated in step 708.

In one embodiment, the first scan plane corresponds to the TTP, the second plane corresponds to the mid-sagittal plane (MSP), the third scan plane corresponds to the trans-cerebellar plane (TCP) and the fourth scan plane corresponds to the trans-ventricular plane (TVP). In another embodiment, the first scan plane corresponds to the TVP, the second scan plane corresponds to the TTP, the third scan plane corresponds to the MSP and the fourth scan plane corresponds to the TCP. In yet another embodiment, the first scan plane corresponds to the TCP, the second scan plane corresponds to the MSP, the third scan plane corresponds to the TTP and the fourth scan plane corresponds to the TVP. In a further embodiment, the first scan plane corresponds to the MSP, the second scan plane corresponds to the TCP, the third scan plane corresponds to the TTP and the fourth scan plane corresponds to the TVP.

Specifically, in one embodiment, determining the MSP includes segmenting the TTP to detect a midline of the cranium and a TTP midpoint based on the segmented TTP and determining a plane parameter vector corresponding to the MSP based on the midline. Further, the method also includes generating the MSP from the determined plane parameter. Further, in another embodiment, determining the TVP includes generating a plurality of TVP candidates, wherein each of the plurality of TVP candidates is parallel to the optimal TTP and orthogonal to the MSP. The step of determining the TVP also includes receiving the second deep learning network configured to determine an optimal TVP and estimating an optimal TVP by processing the plurality of TVP candidates by the second deep learning network.

In a further embodiment, determining the TCP includes generating a plurality of TCP candidates. Each of the plurality of TCP candidates is orthogonal to the optimal MSP and oriented to a perpendicular to the optimal TTP by angle within a predetermined angular span. The step of determining the TCP also includes estimating an optimal TCP by processing the plurality of TVP candidates by the second deep learning network. The second learning network is further configured to determine an optimal TCP. In embodiments, when the, the step of determining the TCP is available before TTP, a search for a parallel plane is initiated among a plurality of TTP candidates to determine the TTP.

Further, the method includes determining a biometric parameter corresponding to nervous system of the fetus based on at least one of the MSP, TCP and TVP and a geometric constraint using a third deep learning network as illustrated in step 710. In one embodiment, the step 710 of determining the biometric parameter includes determining at least one of a head circumference (HC), a biparietal diameter (BPD), an occipito-frontal diameter (OFD), a trans-cerebellar diameter (TCD), a cisterna magna (CM), and a posterior ventricle (Vp) based on one or more of the optimal TTP, the MSP, the TCP and the TVP.

The method also includes determining a nervous system condition of the fetus based on the biometric parameter as in step 712. In one embodiment, determining the nervous system condition includes comparing the biometric parameter with a pre-determined threshold and choosing an option corresponding to the nervous system based on the comparison. In another embodiment, determining the nervous system condition includes determining an object segmentation using a fourth deep learning network, wherein the fourth deep learning network is trained using a plurality of annotated images. In yet another embodiment, determining the nervous system condition includes identifying a location on the objected segmented image and performing automated measurements using a caliper placement algorithm.

It is to be understood that not necessarily all such objects or advantages described above may be achieved in accordance with any particular embodiment. Thus, for example, those skilled in the art will recognize that the systems and techniques described herein may be embodied or carried out in a manner that achieves or improves one advantage or group of advantages as taught herein without necessarily achieving other objects or advantages as may be taught or suggested herein.

While the technology has been described in detail in connection with only a limited number of embodiments, it should be readily understood that the specification is not limited to such disclosed embodiments. Rather, the technology can be modified to incorporate any number of variations, alterations, substitutions or equivalent arrangements not heretofore described, but which are commensurate with the spirit and scope of the claims. Additionally, while various embodiments of the technology have been described, it is to be understood that aspects of the specification may include only some of the described embodiments. Accordingly, the specification is not to be seen as limited by the foregoing description but is only limited by the scope of the appended claims.

1. A method, comprising:
obtaining an initial estimate of a first scan plane corresponding to a fetus of a maternal subject using a first deep learning network during a guided scanning procedure, wherein the scan plane comprises one of a trans thalamic plane (TTP), a trans-ventricular plane (TVP), a mid-sagittal plane (MSP) or a trans-cerebellar plane (TCP);
receiving a three-dimensional (3D) ultrasound volume of the fetus corresponding to the initial estimate of the first scan plane;
determining an optimal first scan plane from the first deep learning network based on the 3D ultrasound volume and the initial estimate of the first scan plane;
determining at least one of a second scan plane, a third scan plane or a fourth scan plane based on the 3D ultrasound volume, the optimal first scan plane and at least one of a clinical constraint corresponding to the TTP, the TVP, the MSP or the TCP using a corresponding second deep learning network, wherein the at least one of the second scan plane, the third scan plane, or the fourth scan plane comprises one of the TTP, the TVP, the MSP or the TCP and is distinctly different from the first scan plane;
determining a biometric parameter corresponding to nervous system of the fetus based on at least one of the first scan plane, the second scan plane, the third scan plane, or the fourth scan plane and the clinical constraint using a third deep learning network; and
determining a nervous system condition of the fetus based on the biometric parameter.

2. The method of claim 1, wherein determining the optimal first scan plane comprises:
generating a plurality of TTP candidates based on the initial estimate;
determining a quality score corresponding to each of the plurality of TTP candidates using the first deep learning network to generate a plurality of quality scores;
determining a minimum score among the plurality of quality scores; and
selecting a TTP candidate among the plurality of TTP candidates corresponding to the minimum score as an optimal TTP.

3. The method of claim 2, wherein determining the second scan plane comprises:
segmenting the optimal TTP to detect a midline falx and a TTP midpoint based on the segmented TTP;
determining a plane parameter vector corresponding to the MSP based on the midline falx; and
generating an optimal MSP from the determined plane parameter.

4. The method of claim 3, wherein determining the third scan plane comprises:
generating a plurality of TVP candidates, wherein each of the plurality of TVP candidates is parallel to the optimal TTP and orthogonal to the MSP;
determining an optimal TVP by using the second deep learning network; and
estimating an optimal TVP by processing the plurality of TVP candidates by the second deep learning network.

5. The method of claim 4, wherein determining the fourth scan plane comprises:
generating a plurality of TCP candidates, wherein each of the plurality of TCP candidates is orthogonal to the optimal MSP and oriented to a perpendicular to the optimal TTP by angle within a predetermined angular span; and
estimating an optimal TCP by processing the plurality of TVP candidates by the second deep learning network, wherein the second learning network is further configured to determine an optimal TCP.

6. The method of claim 5, wherein determining the biometric parameter comprises determining at least one of a head circumference (HC), a biparietal diameter (BPD), an occipito-frontal diameter (OFD), a trans-cerebellar diameter (TCD), a dimension related to cisterna magna (CM), hemisphere (HEM) and nuchal fold (NF), anterior ventricle (Va), cavum septi pelucidi (CSP) or a posterior ventricle (Vp) based on one or more of the optimal TTP, the optimal MSP, the optimal TCP or the optimal TVP.

7. The method of claim 1, wherein determining the nervous system condition comprises comparing the biometric parameter with a pre-determined threshold and choosing an option corresponding to the nervous system based on the comparison.

8. The method of claim 7, wherein determining the nervous system condition comprises performing image segmentation and determining an object in the segmented image using a fourth deep learning network, wherein the fourth deep learning network is trained using a plurality of annotated images.

9. The method of claim 8, wherein determining the nervous system condition comprises identifying a location of the object and performing automated measurements using a caliper placement algorithm.

10. A system, comprising:
an ultrasound scanning probe configured to obtain an initial estimate of a first scan plane corresponding to a fetus of a maternal subject using a first deep learning network during a guided scanning procedure, wherein the scan plane comprises one of a trans thalamic plane (TTP), a trans-ventricular plane (TVP), a mid-sagittal plane (MSP) or a trans-cerebellar plane (TCP);
a data acquisition unit communicatively coupled to the ultrasound probe and configured to receive scan data obtained by the ultrasound scanning probe;
a learning unit communicatively coupled to the data acquisition unit and configured to:
receive, from the data acquisition unit, a three-dimensional (3D) ultrasound volume of the fetus corresponding to the initial estimate of the first scan plane;
determine an optimal scan plane from the first deep learning network based on the 3D ultrasound volume and the initial estimate of the first scan plane;
determine at least one of a second scan plane, a third scan plane or a fourth scan plane based on the 3D ultrasound volume, the optimal first scan plane and at least one of a clinical constraint corresponding to the TTP, the TVP, the MSP or the TCP using a corresponding second deep learning network, wherein the second scan plane, the third scan plane or the fourth scan plane comprises one of the TTP, the TVP, the MSP or the TCP and distinctly different from the first scan plane;
determine a biometric parameter corresponding to nervous system of the fetus based on at least one of the first scan plane, the second scan plane, the third scan plane, or the fourth scan plane and the clinical constraint using a third deep learning network; and
a diagnosis unit communicatively coupled to the learning unit and configured to determine a nervous system condition of the fetus based on the biometric parameter.

11. The system of claim 10, wherein the learning unit is configured to:
generate a plurality of TTP candidates based on the initial estimate;
determine a quality score corresponding to each of the plurality of TTP candidates using the first deep learning network to generate a plurality of quality scores;
determine a minimum score among the plurality of quality scores; and
select a TTP candidate among the plurality of TTP candidates corresponding to the minimum score as an optimal TTP.

12. The system of claim 11, wherein the learning unit is configured to:
segment the optimal TTP to detect a midline of cranium and a TTP midpoint based on the segmented TTP;
determine a plane parameter vector corresponding to the MSP based on the midline; and
generate the MSP from the determined plane parameter as an optimal MSP.

13. The system of claim 12, wherein the learning unit is configured to:
generate a plurality of TVP candidates, wherein each of the plurality of TVP candidates is parallel to the optimal TTP and orthogonal to the MSP;
receive the second deep learning network configured to determine an optimal TVP; and
estimate an optimal TVP by processing the plurality of TVP candidates by the second deep learning network.

14. The system of claim 13, wherein the learning unit is configured to:
generate a plurality of TCP candidates, wherein each of the plurality of TCP candidates is orthogonal to the optimal MSP and oriented to a perpendicular to the optimal TTP by angle within a predetermined angular span; and
estimate an optimal TCP by processing the plurality of TVP candidates by the second deep learning network, wherein the second learning network is further configured to determine an optimal TCP.

15. The system of claim 14, wherein the learning unit is configured to determine at least one of a head circumference (HC), a biparietal diameter (BPD), an occipito-frontal diameter (OFD), a trans-cerebellar diameter (TCD), a cisterna magna (CM), and a posterior ventricle (Vp) based on one or more of the optimal TTP, the optimal MSP, the optimal TCP and the optimal TVP.

16. The system of claim 10, wherein the diagnosis unit is configured to compare the biometric parameter with a pre-determined threshold and choose an option corresponding to the nervous system based on the comparison.

17. The system of claim 16, wherein the diagnosis unit is configured to perform an image segmentation and determine an object in the segmented image using a fourth deep learning network, wherein the fourth deep learning network is trained using a plurality of annotated images.

18. The system of claim 17, wherein the diagnosis unit is configured to identify a location of the object and performing automated measurements using a caliper placement algorithm.

19. A non-transitory computer readable medium having instructions to enable at least one processor unit to:
obtain an initial estimate of a first scan plane corresponding to a fetus of a maternal subject using a first deep learning network during a guided scanning procedure, wherein the scan plane comprises one of a trans thalamic plane (TTP), a trans-ventricular plane (TVP), a mid-sagittal plane (MSP) or a trans-cerebellar plane (TCP);
receive a three-dimensional (3D) ultrasound volume of the fetus corresponding to the initial estimate of the first scan plane;
determine an optimal scan plane from the first deep learning network based on the 3D ultrasound volume and the initial estimate of the first scan plane;
determine at least one of a second scan plane, a third scan plane or a fourth scan plane based on the 3D ultrasound volume, the optimal first scan plane and at least one of a clinical constraint corresponding to the TTP, the TVP, the MSP or the TCP using a corresponding second deep learning network, wherein the second scan plane, the third scan plane or the fourth scan plane comprises one of the TTP, the TVP, the MSP or the TCP and distinctly different from the first scan plane;
determine a biometric parameter corresponding to nervous system of the fetus based on at least one of the first scan plane, the second scan plane, the third scan plane, or fourth scan plane and the clinical constraint using a third deep learning network, and
determine a nervous system condition of the fetus based on the biometric parameter.
